SEQUENCE LISTING (14 sequences)

SEQ ID NO: 1 - DNA, 684 bp, Enterobacteria phage T5
atgagaactgatatactagatagaaaggaagaaatagctcagtggatagctcagggaaaatctaaggcagagatagcaagaatgctatcctgctcctcgaacactttggaagcatatttaggaaaactaggaatagtagttattcctacgaataggcagtatgataataaatataagtctgcaacagaatatttatataacggcagccctatttcttcctataaactaaagaataaaattcttaacgaggggctaaaaccacataagtgtgagagttgcggcttagaaagttggttagacaaaccaattcctttagaactcgaacacaaggatggaaaccactataataatgaatgggataacttagccctcctatgtccaaattgccatgcactaacgcctactcatgcaggcaaaaatattggcaggtatacagaaaggactgtaaatacgtgtgctatttgtcactgcgaaattagctcaagagcaacacactgtaaatcatgtactcctaaagggataactataaatccagatattactgtggagcaaatagagtactgggtatctaaatattcctggattcgtgcctctaaagaactaggattgtctgatactggattaaggaaaaggtataagtccctgacggggaaagatcctaaaagtattaagaaaaatagatga

SEQ ID NO: 2 - protein, 227 aa, Enterobacteria phage T5
MRTDILDRKEEIAQWIAQGKSKAEIARMLSCSSNTLEAYLGKLGIVVIPTNRQYDNKYKSATEYLYNGSPISSYKLKNKILNEGLKPHKCESCGLESWLDKPIPLELEHKDGNHYNNEWDNLALLCPNCHALTPTHAGKNIGRYTERTVNTCAICHCEISSRATHCKSCTPKGITINPDITVEQIEYWVSKYSWIRASKELGLSDTGLRKRYKSLTGKDPKSIKKNR

SEQ ID NO: 3 - DNA, 30 bp, Salmonella heidelberg
taggtactggacttaaaattcaggttttgt

SEQ ID NO: 4 - DNA, 30 bp, Artificial Sequence
  5′ PCR primer for cloning HegA
atgagaactgatatactagatagaaaggaa

SEQ ID NO: 5 - DNA, 30 bp, Artificial Sequence
  3′ PCR primer for cloning HegA
tcatctatttttcttaatacttttaggatc

SEQ ID NO: 6 - DNA, 68 bp, Artificial Sequence
  Sense PCR primer for producing pBS314
aactcgagaatatttaggtactggacttaaaattcaggttttgtgatatcgcgttggccgattcatta

SEQ ID NO: 7 - DNA, 67 bp, Artificial Sequence
  Antisense PCR primer for producing pBS314
aactcgagaatatttaggtactggacttaaaattcaggttttgtgatatccgcgtcagcgggtgttg

SEQ ID NO: 8 - DNA, 34 bp, Artificial Sequence
  PCR Primer xylA-A
ttgctcttccatgcaagcctattttgaccagctc

SEQ ID NO: 9 - DNA, 33 bp, Artificial Sequence
  PCR primer xylA-C
ttgctcttcgttatttgtcgaacagataatggt

SEQ ID NO: 10 - DNA, 60 bp, Artificial Sequence
  PCR Primer
attacgacatcatccatcacccgcggcattacctgattatggagttcaatcggctaactg

SEQ ID NO: 11 - DNA, 60 bp, Artificial Sequence
  PCR Primer
tgcccggtatcgctaccgataaccgggccaacggactgcacagttagccgcagttagccg

SEQ ID NO: 12 - DNA, 7306 bp, Artificial Sequence
  pACHegA vector construct
gtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcaggagggtacctatatgagaactgatatactagatagaaaggaagaaatagctcagtggatagctcagggaaaatctaaggcagagatagcaagaatgctatcctgctcctcgaacactttggaagcatatttaggaaaactaggaatagtagttattcctacgaataggcagtatgataataaatataagtctgcaacagaatatttatataacggcagccctatttcttcctataaactaaagaataaaattcttaacgaggggctaaaaccacataagtgtgagagttgcggcttagaaagttggttagacaaaccaattcctttagaactcgaacacaaggatggaaaccactataataatgaatgggataacttagccctcctatgtccaaattgccatgcactaacgcctactcatgcaggcaaaaatattggcaggtatacagaaaggactgtaaatacgtgtgctatttgtcactgcgaaattagctcaagagcaacacactgtaaatcatgtactcctaaagggataactataaatccagatattactgtggagcaaatagagtactgggtatctaaatattcctggattcgtgcctctaaagaactaggattgtctgatactggattaaggaaaaggtataagtccctgacggggaaagatcctaaaagtattaagaaaaatagatgactgcaggcatgctaaggaggttataaaaaatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatgacgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatgcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcg

SEQ ID NO: 13 - DNA, 3224 bp, Artificial Sequence
  pBS322 target plasmid
ctagattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctctctagccccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagctgtagggataacagggtaatatttaaatagcggataacaatttcacacaggattgcaggtcgagaatatttaggtactggacttaaaattcaggttttgtgatatccgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgatatcacaaaacctgaattttaagtccagtacctaaatattctcgactctagggtcgtgactgggaaaaccctggcggtttaaactgccatttcattacctctttctccgcacccgacatagatgacagcttatcactgatcagtgaattaatggcgatgacgcatcctcacgataatatccgggtaggcgcaatcactttcgtctctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagag

SEQ ID NO: 14 - DNA, 3183 bp, Artificial Sequence
  pBS325 target plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgacagctgtctcttatacacatctcaaccatcatcgatgaattttctcgggtgttctcgcatattggctcgaattcgagctcggtacccggggatcctctagagtcgagaatatttaggtactggacttaaaattcaggttttgtgatatccgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgatatcacaaaacctgaattttaagtccagtacctaaatattctcgacctgcaggcatgcaagcttgccaacgactacgcactagccaacaagagcttcagggttgagatgtgtataagagacagctgtcttaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2007/0178490 A1
(43) Pub. Date: Aug. 2, 2007

(54) HEGA ENDONUCLEASE

(76) Inventor: Frederick R. Blattner, Madison, WI (US)

Correspondence Address: HOWREY LLP, C/O IP DOCKETING DEPARTMENT, 2941 FAIRVIEW PARK DR, SUITE 200, FALLS CHURCH, VA, 22042-2924, US

(21) Appl. No.: 11/551,970

(22) Filed: Oct. 23, 2006

Related U.S. Application Data
(60) Provisional application No. 60/728,982, filed on Oct. 21, 2005.

(51) Int. Cl. C12Q 1/68; C07H 21/04; C12P 21/06; C12N 9/22; C12N 15/09

(52) U.S. Cl. 435/6; 435/69.1; 435/199; 435/320.1; 435/325; 536/23.2

(57) ABSTRACT

An endonuclease which recognizes a specific nucleotide sequence, a gene coding for the endonuclease, a recombinant vector comprising the gene, a host cell comprising the vector, a process for producing the endonuclease, and methods of using the endonuclease are described.

### CROSS REFERENCE TO RELATED APPLICATIONS

This application claims the benefit of U.S. Provisional Patent Application No. 60/728,982, filed Oct. 21, 2005, which is incorporated herein by reference.

### BACKGROUND OF THE INVENTION

Endonucleases are routinely used in current molecular biology protocols, such as the cloning and analysis of genes. An endonuclease may act by recognizing and binding to a particular sequences of nucleotides, also known as the “recognition sequence” or “cognate site,” along a nucleic acid. Once bound, the endonuclease may cleave the molecule within, or to one side of, the recognition sequence by hydrolyzing a phosphodiester bond of the nucleic acid. Different endonucleases may have affinity for different recognition sequences.

There is an ongoing need to obtain recombinant endonucleases because endonucleases recognizing specific recognition sequences may be useful tools, for example, for creating recombinant nucleic acid molecules. The HegA enzyme (SEQ ID NO: 2) is a double stranded endonuclease encoded by the TflIV gene of E. coli bacteriophage T5 (SEQ ID NO: 1). See Akulenko et al., Mol Biol (Moscow) 38:632-41 (2004). The HegA enzyme recognizes a specific 30 base recognition site (SEQ ID NO: 3). The HegA enzyme has been expressed in an in vitro translation system, but had not been cloned or expressed in a host cell.

### SUMMARY OF THE INVENTION

A HegA polypeptide is provided. The HegA polypeptide may comprise the sequence set forth in SEQ ID NO: 2 or a sequence substantially identical thereto or having conservative substitutions that allow the polypeptide to retain its ability to recognize the HegA recognition site.

Also provided is a nucleic acid encoding HegA. The nucleic acid may encode a polypeptide comprising the sequence set forth in SEQ ID NO: 2 or a sequence substantially identical thereto. The nucleic acid may comprise the sequence set forth in SEQ ID NO: 1 or a sequence substantially identical thereto or which codes for the HegA polypeptide by way of degenerate codons.

Also provided is a nucleic acid, comprising a HegA recognition site capable of being cleaved by the HegA polypeptide. The HegA recognition site may comprise the sequence set forth in SEQ ID NO: 3 or a sequence substantially identical thereto.

Also provided is a vector, comprising the nucleic acid encoding HegA. The vector may be a cloning vector. The vector may also be an expression vector wherein the nucleic acid encoding HegA is operatively linked to a promoter. The vector may comprise the sequence set forth in SEQ ID NO: 12 or a sequence substantially identical thereto or a sequence which encodes HegA by way of degenerate codon.

Also provided is a host cell, comprising the vector that comprises the nucleic acid encoding HegA. The vector may be a cloning or expression vector. Also provided is a host cell comprising the HegA recognition sequence. The recognition sequence may be present on a vector. The recognition sequence may also be present on a chromosome of the host cell.

Also provided is a method of producing the HegA polypeptide. The host cell comprising the vector comprising the nucleic acid encoding HegA may be cultured under conditions that allow for expression of the HegA polypeptide.

Also provided is a method of cleaving a HegA recognition sequence. A target nucleic acid sequence comprising the recognition sequence and a HegA polypeptide are provided, whereby the HegA polypeptide cleaves the target nucleic acid. The cleavage may occur in vitro. The cleavage may also occur in vivo, such as in a host cell or organism. The HegA polypeptide may be provided by expressing the nucleic acid encoding the HegA polypeptide either in vitro or in vivo.

Also provided is a method for site directed homologous recombination in a host cell. A host cell is provided comprising a first nucleic acid and a target nucleic acid comprising the HegA recognition sequence. The first nucleic acid and the target nucleic acid may comprise one or more homologous sequences. The target nucleic acid may be cleaved by the HegA polypeptide, whereby homologous recombination may occur between the first nucleic acid and the target nucleic acid. The first nucleic acid and the target nucleic acid may each be either a plasmid or a chromosome of the host cell. The first nucleic acid and the target nucleic acid may be on the same plasmid. The first nucleic acid and the target nucleic acid may also be on the same chromosome.

Also provided is a method of inserting a nucleic acid into a target nucleic acid of a host cell. A host cell is provided comprising a first nucleic acid and a target nucleic acid. The first nucleic acid may comprise a second nucleic acid to be inserted into the target nucleic acid. The target nucleic acid may comprise a HegA recognition sequence. The first nucleic acid and the target nucleic acid may comprise one or more homologous sequences. The second nucleic acid may be proximal to the homologous sequence of the first nucleic acid. Site-directed homologous recombination may be induced between the first nucleic acid and the target nucleic acid, whereby the second nucleic acid is inserted into the target nucleic acid. The second nucleic acid may encode a polypeptide.

Also provided is a method of deleting a nucleic acid from a target nucleic acid of a host cell. A host cell is provided comprising a first nucleic acid and a target nucleic acid. The target nucleic acid may comprise a second nucleic acid proximal to the HegA recognition sequence. The first nucleic acid and the target nucleic acid may comprise one or more homologous sequences. The second nucleic acid may be proximal to the homologous sequence of the target nucleic acid. Site-directed homologous recombination may be induced between the first nucleic acid and the target nucleic acid, whereby the second nucleic acid is deleted from the target nucleic acid. The second nucleic acid may encode a polypeptide.

Also provided is a method of providing a host cell, said host cell comprising a chromosome and an episomal nucleic acid, said chromosome comprising one or more HegA recognition sequences and wherein said episomal nucleic acid lacks a HegA site; providing to the host cell a vector as described herein whereby expression of said vector results in a the production of a HegA polypeptide and wherein said chromosome is degraded by said HegA polypeptide; and isolating said episomal nucleic acid from said host cells.

### BRIEF DESCRIPTION OF THE DRAWINGS

FIG. 1 shows a recognition site for HegA (SEQ ID NO: 3) and the location of cleavage.

FIG. 2 shows a map of the pACHegA construct (SEQ ID NO: 12).

FIG. 3 shows a map of the pBS322 target plasmid (SEQ ID NO: 13).

FIG. 4 shows a map of the pBS325 target plasmid (SEQ ID NO: 14).

FIG. 5 demonstrates the HegA transposon strategy.

### DETAILED DESCRIPTION

The endonuclease HegA has been successively cloned for the first time. Moreover, HegA has for the first time been expressed in a host cell and shown to be active in vivo without detriment to the host cell.

1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

“Clone” used in reference to an insert sequence and a vector may mean ligation of the insert sequence into the vector or its introduction by recombination either homologous, site specific or illegitimate as the case may be. When used in reference to an insert sequence, a vector, and a host cell, the term may mean to make copies of a given insert sequence. The term may also refer to a host cell carrying a cloned insert sequence, or to the cloned insert sequence itself.

“Complement,” “complementary” or “complementarity” used herein may mean Watson-Crick or Hoogsteen base pairing between nucleotides or nucleotide analogs of nucleic acid molecules. For example, the sequence 5′-A-G-T-3′ is complementary to the sequence 3′-T-C-A-5′. Complementarity may be “partial,” in which only some of the nucleotides are matched according to the base pairing rules. Or, there may be “complete” or “total” complementarity between the nucleic acids. The degree of complementarity between nucleic acid strands may have effects on the efficiency and strength of hybridization between nucleic acid strands.

“Encoding” or “coding” used herein when referring to a nucleic acid may mean a sequence of nucleotides, which upon transcription into RNA and subsequent translation leads to the synthesis of a given protein, polypeptide, peptide or amino acid sequence. Such transcription and translation may actually occur in vitro or in vivo, or may be strictly theoretical based on the standard genetic code.

“Enzyme” used herein may mean a protein which acts as a catalyst to induce a chemical change in other compounds, thereby producing one or more products from one or more substrates. Enzymes are referred to herein using standard nomenclature or by their EC number, as recommended by the Nomenclature Committee of the International Union of Biochemistry and Molecular Biology as of Mar. 11, 2004.

“Expression control sequence” used herein may mean a promoter or array of transcription factor binding sites that direct transcription of a nucleic acid operatively linked thereto.

“Free end” used in reference to a double-stranded nucleic acid may mean a linear nucleic acid with blunt free ends or sticky free ends, or a combination thereof.

“Gene” used herein may refer to a nucleic acid (e.g., DNA or RNA) that comprises a nucleic acid sequence encoding a polypeptide or precursor thereto. The polypeptide can be encoded by a full length coding sequence or by any portion of the coding sequence so long as the desired activity or functional properties (e.g., enzymatic activity, ligand binding, signal transduction, antigenicity etc.) of the full-length or fragment are retained. The term also encompasses the sequences located adjacent to the coding region on both the 5′ and 3′ ends that contribute to the gene being transcribed into a full-length mRNA. The term “gene” encompasses both cDNA and genomic forms of a gene. A genomic form or clone of a gene may contain the coding region interrupted with non-coding sequences termed (e.g., introns).

“Host cell” used herein may be a naturally occurring cell or a transformed cell that may contain a vector and may support replication of the vector. Host cells may be cultured cells, explants, cells in vivo, and the like. Host cells may be prokaryotic cells such as E. coli, or eukaryotic cells such as yeast, insect, amphibian, or mammalian cells, such as CHO and HeLa.

“Identical” or “identity” used herein in the context of two or more nucleic acids or polypeptide sequences, may mean that the sequences have a specified percentage of residues that are the same over a region of comparison. The percentage may be calculated by optimally aligning the two sequences, comparing the two sequences over the specified region, determining the number of positions at which the identical residue occurs in both sequences to yield the number of matched positions, dividing the number of matched positions by the total number of positions in the region of comparison, and multiplying the result by 100 to yield the percentage of sequence identity. In cases where the two sequences are of different lengths or the alignment produces one or more staggered ends and the specified region of comparison includes only a single sequence, the residues of single sequence may be included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Introduce” or “introduced” when used in reference to adding a nucleic acid to a strain, may mean that the nucleic acid may be integrated into the chromosome of the strain or contained on a vector, such as a plasmid, in the strain.

“Nucleic acid” used herein may mean any nucleic acid containing molecule including, but not limited to, DNA or RNA. The depiction of a single strand also defines the sequence of the complementary strand. Thus, a nucleic acid also encompasses the complementary strand of a depicted single strand.

A nucleic acid may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid may include any base analogs of DNA and RNA including, but not limited to, 4-acetylcytosine, 8-hydroxy-N6-methyladenosine, aziridinylcytosine, pseudoisocytosine, 5-(carboxyhydrdxylmethyl)uracil, 5-fluorouracil, 5-bromouracil, 5-carboxymethylaminomethyl-2-thiouracil, 5 carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracils, 5-methoxyaminomethyl-2-thiouracil, ÿ-D-maninosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

“Nucleotide” used herein may refer to a monomeric unit of nucleic acid (e.g. DNA or RNA) consisting of a pentose sugar moiety, a phosphate group, and a nitrogenous heterocyclic base. The base may be linked to the sugar moiety via the glycosidic carbon (1′ carbon of the pentose). The combination of base and sugar may be called a nucleoside. When the nucleoside contains a phosphate group bonded to the 3′ or 5′ position of the pentose, it may be referred to as a nucleotide. A sequence of operatively linked nucleotides may be referred to as a “base sequence” or “nucleotide sequence” or “nucleic acid sequence,” and may be represented herein by a formula whose left to right orientation is in the conventional direction of 5′-terminus to 3′-terminus.

“Operably linked” used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Overexpressing” used herein may mean that the total cellular activity of protein encoded by a gene is increased. The total cellular activity of a protein may be due to increased cellular amounts of a protein, or increased half-life of the protein. Total cellular amounts of a protein may be increased by methods including, but not limited to, amplification of the gene coding said protein or operatively linking a strong promoter to the gene coding said protein.

“Promoter” used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Protein” used herein may mean a peptide, polypeptide and protein, whether native or recombinant, as well as fragments, derivatives, homologs, variants and fusions thereof.

“Region of comparison” used herein when referring to a genome may be 1×10⁷, 1.5×10⁷, 2×10⁷, 2.5×10⁷, 3×10⁷, 3.5×10⁶, 4×10⁷ or more nucleotides or base pairs, and when referring to a nucleic acid or polypeptide sequence may be 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 250, 500, 10³, 5×10³, 10⁴, 5×10⁴, 10⁵, 5×10⁵, 10⁶ or more residues. The region of comparison may also be the full length of one or both of the comparison sequences.

“Selectable marker” used herein may mean any gene which confers a phenotype on a host cell in which it is expressed that may be used to facilitate the identification and/or selection of cells which are transfected or transformed with a genetic construct. Representative examples of selectable markers include the ampicillin-resistance gene (Amp^{r}), tetracycline-resistance gene (Tc^{r}), bacterial kanamycin-resistance gene (Kan^{r}), zeocin resistance gene, the AURI-C gene which confers resistance to the antibiotic aureobasidin A, phosphinothricin-resistance gene, neomycin phosphotransferase gene (nptII), hygromycin-resistance gene, beta-glucuronidase (GUS) gene, chloramphenicol acetyltransferase (CAT) gene, green fluorescent protein (GFP)-encoding gene and luciferase gene.

“Stringent hybridization conditions” used herein may mean conditions under which a first nucleic acid sequence (e.g., probe) will hybridize to a second nucleic acid sequence (e.g., target), such as in a complex mixture of nucleic acids, but to no other sequences above background levels. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 533 SSC, 1% SDS, incubating at 65° C., with wash in 0.2×SSC, and 0.1% SDS at 65° C.

“Substantially complementary” used herein may mean that a first sequence is at least 60%, 65%, 70%, 75%, 80%, 85%, 90%, 95%, 97%, 98% or 99% identical to the complement of a second sequence over a region of comparison, or in the case of nucleic acids, that the two sequences hybridize under stringent hybridization conditions.

“Substantially identical” used herein may mean that a first and second sequence are at least 60%, 65%, 70%, 75%, 80%, 85%, 90%, 95%, 97%, 98% or 99% identical over a region of comparison, or in the case of nucleic acids, if the first sequence is substantially complementary to the complement of the second sequence.

“Vector” used herein may mean a nucleic acid molecule capable of transporting another nucleic acid to which it has been linked. Representative examples of a vector include, but not limited to, a plasmid, cosmid, phage, phagemid, BAC, YAC or viral vector.

2. HegA

a. Nucleic Acid

A nucleic acid encoding HegA is provided. The nucleic acid may comprise the sequence set forth in SEQ ID NO: 1 or a sequence substantially identical thereto. The sequence of the nucleic acid may be changed, for example, to account for codon preference in a particular host cell. The nucleic acid may be synthesized or derived from bacteriophage T5 DNA, such as ATCC #11303-B5, using standard molecular biology techniques.

Also provided is a nucleic acid comprising a HegA recognition site. A HegA recognition site may comprise the sequence set forth in SEQ ID NO: 3 or a sequence substantially identical thereto.

b. Polypeptide

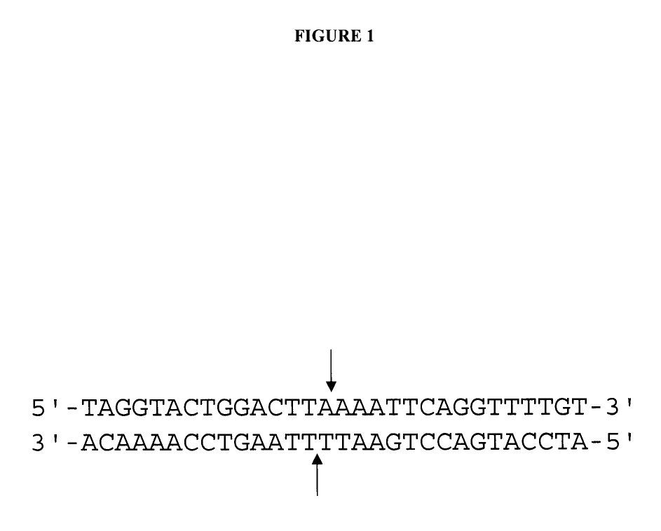

Also provided is a HegA polypeptide. The HegA polypeptide may comprise the sequence set forth in SEQ ID NO: 2 or a sequence substantially identical thereto. The HegA polypeptide may be an endonuclease that cleaves a HegA recognition sequence. The recognition sequence may be cleaved as indicated in FIG. 1.

The HegA polypeptide may be a fusion protein comprising a polypeptide or peptide which may be used to purify the HegA polypeptide. Representative examples of such peptides include a histidine tag a maltose-binding protein fusion or a chitin-binding intein fusion.

c. Synthetic Gene

Also provided is a synthetic gene comprising the HegA nucleic acid operably linked to a transcriptional and/or translational regulatory sequence. The synthetic gene may be capable of expressing the HegA polypeptide. The synthetic gene may also comprise terminators at the 3′-end of the transcriptional unit of the synthetic gene sequence. The synthetic gene may also comprise a selectable marker.

d. Vector

Also provided is a vector comprising the HegA nucleic acid or synthetic HegA gene. The vector may be a cloning vector. The vector may also be an expression vector.

Also provided is a vector comprising the HegA recognition site. The vector may comprise a nucleic acid of interest with the HegA recognition site within or adjacent to the nucleic acid of interest. The nucleic acid may encode a polypeptide.

The vector may also comprise additional elements. The vector may also comprise a selectable marker gene to allow the selection of transformed host cells. The vector may also comprise two replication systems allowing it to be maintained in two organisms, e.g., in one host cell for expression and in a second host cell (e.g., bacteria) for cloning and amplification. For integrating expression vectors, the expression vector may comprise a sequence homologous to a host cell genome, such as two homologous sequences which flank the expression construct. The integrating vector may be directed to a specific locus in the host cell by selecting the appropriate homologous sequence for inclusion in the vector.

e. Host Cell

Also provided is a host cell, comprising the HegA vector, synthetic HegA gene or HegA nucleic acid. The host cell may be any cell that is capable of being transformed by the vector, synthetic gene or nucleic acid. The host cell may also be any cell that is capable of expressing the HegA polypeptide. Representative host cells that may be used are available from the American Type Culture Collection.

Also provided is a host cell comprising the HegA recognition site. The host cell may comprise a nucleic acid of interest with the HegA recognition site within or adjacent to the nucleic acid of interest. The nucleic acid may encode a polypeptide. The HegA recognition sequence may be on a vector in the host cell. The HegA recognition sequence may also be on a chromosome of the host cell.

The host cell may be prokaryotic, such as bacterial, or eukaryotic, such as fungal (e.g., yeast), plant, insect, amphibian or animal cell. Representative examples of a bacterial host cell include, but are not limited to, E. coli strains such as K-12 or B. The K-12 strain may be MG1655. The bacterial host cell may also be a reduced genome bacteria. Reduced genome bacteria are discussed in copending U.S. patent application Ser. Nos. 10/057,582 (now U.S. Pat. No. 6,989,245), 10/896,739, 60/634,611 and 60/709,960, 11/275,094, and 11/400,711, which are incorporated herein by reference. Representative examples of reduced genome bacteria strains include, but are not limited to, MDS12, MDS13. MDS39, MDS40, MDS41-R13, MDS41E, MDS42, MDC42recA and MDS43. Representative examples of a mammalian host cell include CHO and HeLa cells.

3. Kit

Also provided is a HegA kit. The kit may comprise the HegA nucleic acid. The kit may also comprise the HegA polypeptide. The kit may also comprise the synthetic HegA gene. The kit may also comprise a vector comprising the HegA nucleic acid. The kit may also comprise a vector comprising the HegA recognition site. The kit may also comprise a host cell capable of expressing the HegA polypeptide. The kit may also comprise a host cell comprising the HegA recognition site.

4. Transforming a Host Cell

Also provided is a method of transforming a host cell with the HegA vector, synthetic HegA gene or HegA nucleic acid. The host cell may be contacted with the vector, synthetic gene or nucleic acid under conditions that allow transformation of the host cell. The host cell may be transformed by methods including transformation, transfection, electroporation, microinjection, or by means of liposomes (lipofection). The transformed cell may be selected, for example, by selecting for a selectable marker on the vector, synthetic gene or nucleic acid.

5. Producing HegA Polypeptide

Also provided is a method of producing the HegA polypeptide. A host cell comprising the HegA vector, synthetic HegA gene or HegA nucleic acid that is capable of expressing HegA may be provided. The host cell may be incubated under conditions that allow expression of the HegA polypeptide. The HegA polypeptide may be purified using standard chromatographic techniques.

6. Cleavage of HegA Recognition Site

Also provided is a method of cleaving the HegA recognition site in a target nucleic acid. A target nucleic acid comprising the HegA recognition site may be contacted with the HegA polypeptide under conditions that allow cleavage of the recognition site. The target nucleic acid may be cleaved in vitro or in vivo. The recognition site may be present in a linear or circular target nucleic acid. The target nucleic acid may be a plasmid or a chromosome. The recognition site may be a naturally occurring site in the target nucleic acid or may be introduced into the target nucleic acid by methods including, but not limited to, mutagenesis (e.g., site-directed or cassette), homologous recombination or transposition.

The ability to cleave HegA recognition sites in vivo without detriment to the host cell allows HegA to be used in a number of techniques for the modification of nucleic acids (e.g., chromosomal and plasmid) within a host cell. For example, HegA may be used to induce the introduction of a double-strand break at a HegA recognition sequence in a target nucleic acid, such as a plasmid or a chromosome. Linear nucleic acids can be degraded by the action of the host RecBCD nuclease and thereby removed from the cell. The double-strand break in the target nucleic acid may also induce homologous recombination within the target nucleic acid (intrastrand homologous recombination) or between the target nucleic acid and another nucleic acid (interstrand homologous recombination). The homologous recombination may lead to the insertion or deletion of a portion of a nucleic acid (e.g., a gene) The nucleic acid may encode a polypeptide. Representative methods of using HegA include the methods for using I-SceI and other endonucleases and their cognate recognition sites as disclosed in U.S. patent application Ser. Nos. 10/057,582, 10/896,739, 10/152,994 and 10/931,246, and U.S. Pat. Nos. 5,474,896, 5,792,632, 5,866,361, 5,948,678, 5,962,327, 6,238,924, 6,395,959, 6,610,545, 6,822,137 and 6,833,252, the contents of which are incorporated herein by reference.

### EXAMPLE 1

### Cloning of HegA

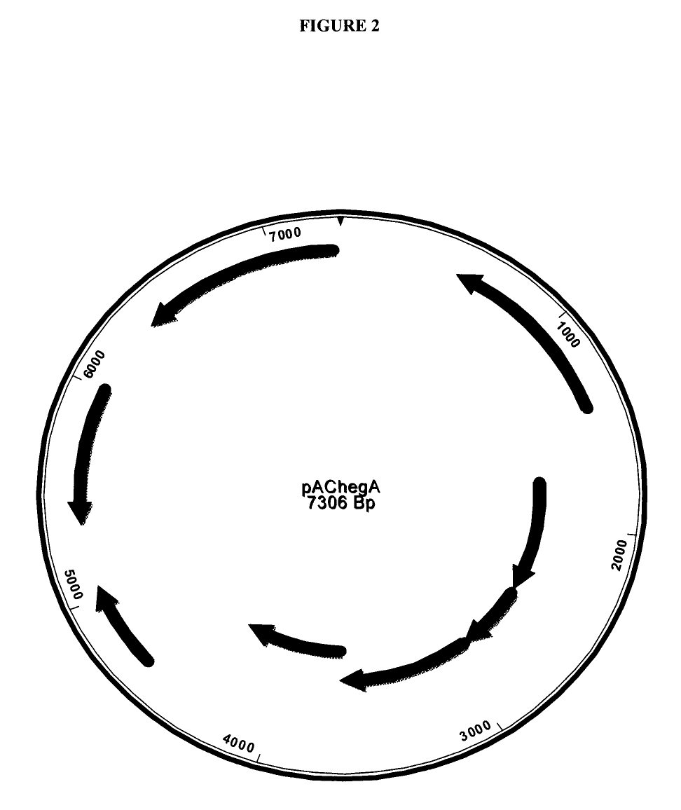

The HegA gene was derived from bacteriophage T5 DNA isolated from ATCC #11303 by PCR amplification. The template DNA was purified by standard phenol chloroform extraction of a plate lysate of the reduced genome E. coli strain MDS42 using the lambda genomic DNA isolation procedure of Sambrook and Russell. Primers were designed to hybridize to regions flanking the HegA gene from the bacteriophage T5 sequence available at NCBI (Accesion number NC_{—}005859). The upstream primer had the sequence 5′-ATGAGAACTGATATACTAGATAGAAAGGAA-3′ (SEQ ID NO: 4). The downstream primer had the sequence 5′-TCATCTATTTTTCTTAATACTTTTAGGATC-3′ (SEQ ID NO: 5). A standard amplification reaction was performed using high-fidelity Pfu polymerase, approximately 10 ng of T5 DNA and 0.5 μM of each primer in 50 μl total volume per manufacturers suggestion (New England Biolabs). A single 684 bp product was produced and ligated to a linear PCR fragment of pACBSR (Herring et al, 2003, Gene 331:153), so that the HegA CDS was between the Lambda red fragment and Ara promoter of the plasmid. The plasmid also possesses a temperature sensitive origin of replication. The final construct (pACHegA) contained the HegA and Lambda gam, beta and exo genes under control of a single araC regulated promoter (FIG. 2).

### EXAMPLE 2

### Construction of HegA Target Plasmids

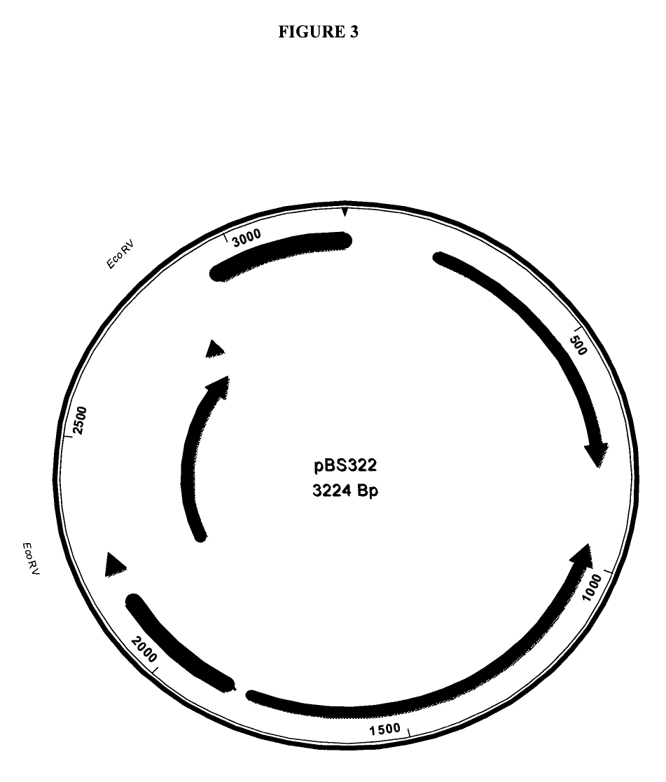

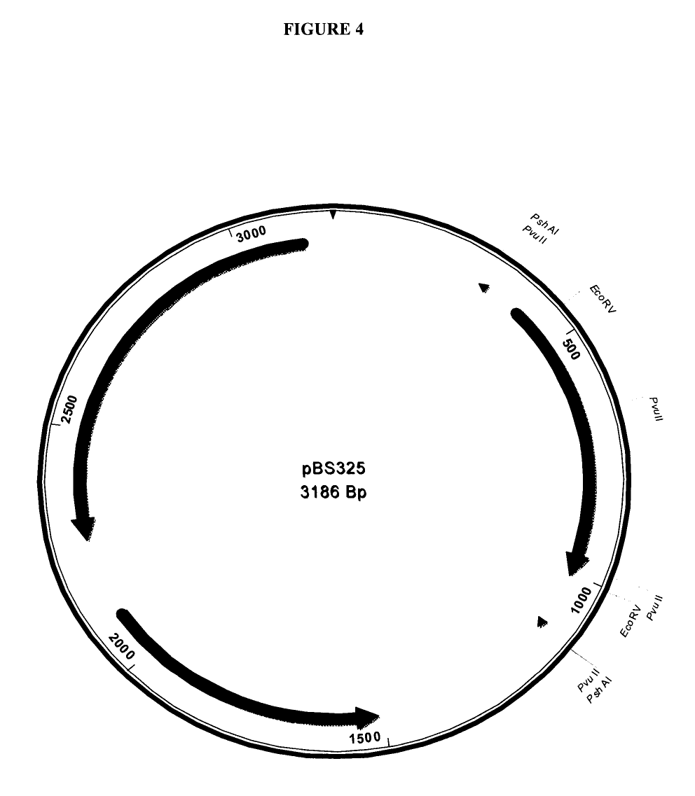

Target plasmids were constructed to test the in vivo endonuclease activity of HegA in E. coli. The first target plasmid was a pUC derivative containing two HegA cognate sites and was designated pBS322 (FIG. 3). The second target plasmid contained two HegA sites within a Tn5 derived cloning vector (Epicentre), and was designated pBS325 (FIG. 4).

To produce the HegA target plasmids, two primers were employed each with EcoRV, HegA, SspI and XhoI sites as well as homology to the alpha fragment of lacZ in pUC19. The sense primer had the sequence 5′-AACTCGAGAATATTTAGGTACTGGACTTAAAATTCA GGTTTTGTGATATCGCGTTGGCCGATTCATTA-3′ (SEQ ID NO: 6). The antisense primer had the sequence 5′-AACTCGAGAATATTTAGGTACTGGACTTAAAATTCAGGTTTTGTG ATATCCGCGTCAGCGGGTGTTG 3′ (SEQ ID NO: 7). A standard amplification reaction was performed using high-fidelity Pfu polymerase, approximately 10 ng of T5 DNA and 0.5 μM of each primer in 50 μl total volume per manufacturers suggestion (New England Biolabs). A single 600 base pair product was gel purified and subsequently treated with Taq polymerase to add 3′ single A overhangs and then cloned into the pGEMT-EZ vector (Promega) to produce pBS314. The cloned PCR fragment was excised from pBS314 as an XhoI fragment and ligated into SalI digested pDF148 (a pUC19 derivative containing an rrnB and a tL3 terminator flanking a unique SalI site) to produce pBS322. Plasmid pBS325 was produced by ligating the XhoI fragment of pBS314 into SalI digested pMOD-2 (Epicentre)

### EXAMPLE 3

### In Vivo Expression and Activity of HegA

In order to determine whether HegA could be expressed in vivo and cleave its cognate site without being toxic to an E. coli host cell, MDS42 cells were cotransformed with pACHegA and either one of the HegA cognate site containing plasmids, pBS322 and pBS325.

Cells containing pACHegA together with pBS322 or pBS325 are resistant to chloramphenicol (by virtue of the CAT gene of pACHegA) and ampicillin (by virtue of the bla gene of pBS322 or PBS325). When exposed to arabinose, induction of the Para promoter of the pACHegA plasmid produces HegA endonuclease, which if active will cleave the cognate sites in pBS322 or pBS325 as well as any recognition site present in the host cell chromosome. Linearizing pBS322 or pBS325 in vivo results in loss of ampicillin resistance due to RecBCD-mediated degradation of the linearized plasmid within the cell.

The in vivo activity of the HegA enzyme can be scored by the frequency with which ampicillin resistance is lost on induction of the ara promoter. The lethality of the HegA enzyme to the host cell can be scored by the frequency with which cell numbers are reduced upon induction of the ara promoter. As shown in the Table 1 below, induction with arabinose resulted in a significant loss of ampicillin resistance. At the same time, induction with arabinose was not toxic to the host cells. The apparent lack of complete loss of ampicillin resistance is likely due to target plasmids being based on ligation products. The apparent HegA resistant clones most likely represent vector religation background. These clones are amp resistant but without the presence of a HegA cognate site so that they are insensitive to the presence or absence of HegA.

TABLE 1
Growth of co-transformants (patched
colonies) on LB supplemented with:

| | minus Arabinose | | | plus Arabinose | | |
|---|---|---|---|---|---|---|
| | Amp | cam | amp/cam | amp | cam | amp/cam |
| PACHegA + | 50 | 50 | 50 | 18 | 50 | 18 |
| pBS322 | | | | | | |
| PACHegA + | 8 | 8 | 8 | 1 | 8 | 1 |
| pBS325 | | | | | | |

In addition to allowing degradation of plasmids containing HegA sites, cleavage of any HegA sites present in the host cell chromosome will also result in degradation of the host chromosome, which will eventually result in death of the host cell. Although no HegA recognition sites are present in any sequenced bacterial chromosomes to date, introduction of sequences containing HegA sites into a bacterial chromosome would render such cells inviable once HegA was actively expressed within the cells. Degradation of bacterial chromosomes may, in some cases improve production and/or recovery of episomal nucleic acids, by increasing the availability of nucleotides recycled by chromosome degradation following double strand cleavage of the chromosome by HegA and by removing intermediate molecular weight chromosomal fragments that often co-purify with episomal nucleic acids.

### EXAMPLE 4

### Use of HegA in Gene Gorging

Gene-gorging may involve co-transformation of a host cell with two plasmids. The first plasmid is a HegA expression plasmid, such as pACHegA. The second plasmid contains a desired DNA sequence between two HegA sites. The second plasmid may be generated, for example, by inserting a blunt fragment into EcoRV digested pBS322 with recombinant inserts being recognizable by the loss of lacZ alpha complementation. The desired DNA may have any nucleic acid sequence, but should possess homology to sequence flanking the target region. Using pACHegA and a pBS322 derived plasmid, the host cell may be co-transformed with the two plasmids and plated on permissive media containing sufficient arabinose to induce production of HegA and Lambda Gam, Beta and Exo from pACHegA.

HegA may release the DNA fragment from the pBS322 derivative and the Lambda Gam, Beta, Exo enzymes may facilitate homologous recombination with the target sequence. This target sequence can be chromosomal or a region of another plasmid. The plasmid backbone of the pBS322 plasmid may be eventually degraded by the RecBCD system of the host. The pACHegA plasmid may be cured from the cell, or the chromosomal segment containing the new sequence may be moved to a plasmid free background, by standard methods (e.g., treatment with acradine orange or P1 transduction, respectively).

### EXAMPLE 5

### Heg-Poson Strategy

A recombinant DNA molecule may be constructed containing a unique HegA site and a selectable marker such as an antibiotic resistance gene. A modified Tn5 transposon is such an example. This molecule may be capable of in vitro random transposition and may available with many different antibiotic markers. A HegA site is introduced into the Tn5 derivative by a two-step process beginning with plasmid pMOD2 (Epicentre), which contains a unique SalI site bracketed by IS5 mosaic ends. The plasmid also carries a bla gene and a ColE1 derived origin of replication. The lacZ gene flanked by EcoRV, HegA, Sspl and XhoI sites used to produce pBS322 was introduced into the unique SalI site of pMOD2 to form pBS325. An antibiotic resistance marker, for example the kanamycin resistance gene of pACYC177, is produced by PCR amplification and cloned into the EcoRV digested pBS325. This produces a plasmid possessing a recombinant Tn5-like transposon containing HegA sites and a selectable marker. In the case in which kanamycin resistance is introduced into pBS325 as described, the resulting plasmid is designated pBS364. Digestions of pBS364 with PvuII or PshAI releases the transposon fragment which is gel purified and treated with EZ-Tn5 transposase (Epicentre). The HegA containing transposons can be directly transformed into recipient cells and a random integration library can be constructed in which HegA sites are introduced randomly throughout the genome. The location of individual inserts within the transposon library can be determined by a variety of methods (e.g., outward sequencing) and specific clones identified within the library for applications involving specific genes.

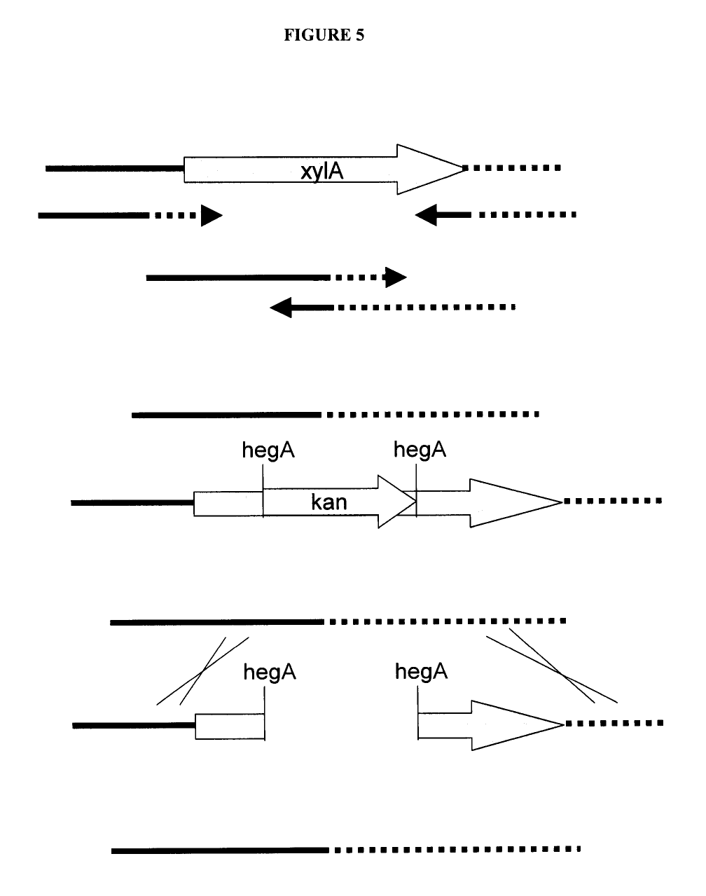

Alternatively, a variant of the transposon method allows directed mutagenesis of any locus within the genome involving PCR amplification of the desired target and in vitro transposition into the target DNA molecule. This introduces the antibiotic resistance marker as well as HegA sites into the target sequence. The in vitro transposition product is then transformed into a host cell expressing Lambda Gam, Beta and Exo to facilitate recombination of the linear target sequence containing the transposon into the genome. A specific example of this last procedure involves introduction of the transposon derived from pBS364 into the xylA locus of E. coli. The 1.3 kb xylA target sequence is produced by PCR amplification of E coli K-12 genomic DNA using primers specific to each side of the gene. Primer xylA-A has the sequence 5′-TTGCTCTTCCATGCAAGCCTATTTTGACCAGCTC-3′ (SEQ ID NO: 8) and primer xylA-C has the sequence 5′-TTGCTCTTCGTTATTTGTCGAACAGATAATGGT-3′ (SEQ ID NO: 9). The in vitro transposition results in a recombinant fragment approximately 2.5 kb in length. The entire in vitro transposition reaction was transformed into the reduced genome E. coli strain MD46 containing pKD46. Recombinational inserts into the chromosomal xylA locus are identified by their inability to ferment xylose on Maconkey media. Complete deletion of the xylA locus is achieved by generating a DNA fragment that encodes the desired deletion junction. In this case, two primers with the sequences 5′-ATTACGACATCATCCATCACCCGCGGCA TTACCTGATTATGGAGTTCAATCGGCTAACTG-3′ (SEQ ID NO: 10) and 5′-TGCCCGGT ATCGCTACCGATAACCGGGCCAACGGACTGCACAGTTAGCCGCAGTTAGCCG-3′ (SEQ ID NO: 11) were designed using the general strategy outlined in FIG. 5. The two primers are annealed and extended using Klenow or any other suitable polymerase to produce a double-stranded DNA fragment. This fragment is co-transformed into the xylA insertion mutant strain with pACHegA and the cells grown in the presence of arabinose. Successful deletion of the transposon region is indicated by loss of kanamycin resistance.

1. An isolated polypeptide comprising the sequence set forth in SEQ ID NO: 2 or a sequence substantially identical thereto.

2. The polypeptide of claim 1 wherein the polypeptide is a fusion protein.

3. An isolated nucleic acid encoding the polypeptide of claim 1.

4. The nucleic acid of claim 3 wherein the nucleic acid comprises the sequence set forth in SEQ ID NO: 1 or a sequence substantially identical thereto.

5. An isolated nucleic acid comprising a HegA recognition site capable of being cleaved by the HegA polypeptide of claim 1.

6. The nucleic acid of claim 5 wherein the recognition site comprises the sequence set forth in SEQ ID NO: 3 or a sequence substantially identical thereto.

7. A vector comprising the nucleic acid of claim 3.

8. The vector of claim 7 wherein the vector is an expression vector comprising a promoter operatively linked to the nucleic acid.

9. The vector of claim 8 wherein the vector comprises the sequence set forth in SEQ ID NO: 12 or a sequence substantially identical thereto.

10. A host cell comprising the vector of claim 7.

11. A host cell comprising the expression vector of claim 8.

12. A host cell comprising the nucleic acid comprising a HegA recognition site of claim 5.

13. The host cell of claim 12 wherein the recognition site is located on a vector.

14. The host cell of claim 12 wherein the recognition site is located on a chromosome of the host cell.

15. A method of producing a HegA polypeptide comprising culturing the host cell of claim 11 under conditions suitable for expression of the HegA polypeptide.

16. A method of cleaving a target nucleic acid comprising a HegA recognition sequence, the method comprising:
(a) providing a target nucleic acid comprising the HegA recognition sequence, and
(b) providing the polypeptide of claim 1, whereby the polypeptide cleaves the target nucleic acid.

17. The method of claim 16 wherein cleavage occurs in vitro.

18. The method of claim 16 wherein cleavage occurs in vivo.

19. The method of claim 18 wherein cleavage occurs in a host cell or organism.

20. A method for site directed homologous recombination in a host cell, comprising:
(a) providing a host cell comprising:
(i) a first nucleic acid; and
(ii) a target nucleic acid comprising a HegA recognition sequence,
wherein the first nucleic acid and target nucleic acid comprise one or more homologous sequences,
(b) cleaving the target nucleic according to the method of claim 19, whereby homologous recombination occurs between the first nucleic acid and the target nucleic acid.

21. The method of claim 20, wherein the first nucleic acid and the target nucleic acid are plasmids.

22. The method of claim 20, wherein the first nucleic acid is a plasmid and the target is a chromosome of the host cell.

23. The method of claim 20, wherein the first nucleic acid and the target nucleic acid are a chromosome of the host cell.

24. The method of claim 23, wherein the first nucleic acid and the target nucleic acid are on the same chromosome.

25. A method of inserting a nucleic acid into a target nucleic acid of a host cell comprising:
(a) providing a host cell comprising:
(i) a first nucleic acid comprising a second nucleic acid to be inserted into the target nucleic acid;
(ii) a target nucleic acid comprising a HegA recognition sequence,
wherein the first nucleic acid and target nucleic acid comprise one or more homologous sequences, and wherein the second nucleic acid is proximal to the homologous sequence of the first nucleic acid; and
(b) inducing site-directed homologous recombination between the first nucleic acid and the target nucleic acid according to the method of claim 20, whereby the second nucleic acid is inserted into the target nucleic acid.

26. The method of claim 25 wherein the second nucleic acid encodes a polypeptide.

27. A method of deleting a nucleic acid from a target nucleic acid of a host cell comprising:
(a) providing a host cell comprising:
(i) a first nucleic acid;
(ii) a target nucleic acid comprising a second nucleic acid proximal to a HegA recognition sequence,
wherein the first nucleic acid and target nucleic acid comprise one or more homologous sequences, and wherein the second nucleic acid is proximal to the homologous sequence of the target nucleic acid; and
(b) inducing site-directed homologous recombination between the first nucleic acid and the target nucleic acid according to the method of claim 20, whereby the second nucleic acid is deleted from the target nucleic acid.

28. The method of claim 27 wherein the second nucleic acid encodes a polypeptide.

29. A method of improving production and/or recovery of episomal nucleic acids from a host cell, the method comprising:
(a) providing a host cell, said host cell comprising a chromosome and an episomal nucleic acid, said chromosome comprising one or more HegA recognition sequences and wherein said episomal nucleic acid lacks a HegA site;
(b) providing to the host cell a vector according to claim 8 whereby expression of said vector results in a the production of a HegA polypeptide and whereby said chromosome is degraded by said HegA polypeptide; and
(c) isolating said episomal nucleic acid from said host cells.

30. The method of claim 29 wherein said HegA recognition sites are inserted into said chromosome.

31. The method of claim 21 wherein said promoter is an inducible promoter.

32. A host cell, wherein the genome of said host cell comprises a HegA recognition site.

33. A host cell comprising a genome, said genome modified to comprise a HegA recognition site.

34. The host cell of claim 32 or 33 wherein said host cell is a bacteria.

35. The host cell of claim 34 wherein said bacteria is E. coli.
